(19) United States
(12) Patent Application Publication
(10) Pub. No.: US 2012/0130245 A1
(43) Pub. Date: May 24, 2012

(54) ULTRASONIC DIAGNOSTIC APPARATUS AND REGION-OF-INTEREST

(75) Inventor: Tomoaki Chono, Tokyo (JP)

(73) Assignee: HITACHI MEDICAL CORPORATION, Chiyoda-ku, Tokyo (JP)

(21) Appl. No.: 13/387,517

(22) PCT Filed: Jul. 28, 2010
(86) PCT No.: PCT/JP2010/062676

§ 371 Date: Jan. 27, 2012

(30) Foreign Application Priority Data
Jul. 30, 2009 (JP) 2009-177208

(51) Int. Cl. A61B 8/00; G06K 9/00

(52) U.S. Cl. 600/443; 382/131

(57) ABSTRACT

Disclose is an ultrasonic diagnostic apparatus which transmits/receives an ultrasonic wave to/from a region including a carotid artery of an object, images an ultrasonic image, and comprises a thickness measurement unit configured to measure the thickness of an intima-media complex from the ultrasonic image, the ultrasonic diagnostic apparatus being provided with a region-of-interest setting unit configured to scan the ultrasonic image and set a region of interest including the intima-media complex on the ultrasonic image on the basis of the degree of concentration of contour slate points of the carotid artery, wherein the thickness measurement unit measures the thickness of the intima-media complex on the basis of boundaries within the set region of interest.

### FIELD OF THE INVENTION

The present invention relates to an ultrasonic diagnostic apparatus and region-of-interest setting method therefore capable of setting a region of interest (ROI) which is a target for measuring intima-media thickness (IMT) in an ultrasonic image of a carotid artery of an imaged object so as to measure an IMT value.

### DESCRIPTION OF RELATED ART

IMT measurement is known as effective for finding arterial sclerosis in an object to be examined. IMT measurement requires the procedure that a medical service worker (an examiner) such as a doctor or a clinical laboratory technologist obtains an ultrasonic image by applying an ultrasonic probe onto a carotid artery part of the object and sets an ROI on the obtained ultrasonic image.

The examiner may set an ROI manually. However, if an examiner executes manual ROI setting operation related to IMT measurement for all of patients, effective image diagnosis cannot be performed.

Given this factor, Patent Document 1 discloses an ROI setting method related to IMT measurement. Patent Document 1 proposes the ROI setting method which sets the position of an artery having the minimum brightness as a lumen, extracts the region close to an ultrasonic probe from the position of lumen as an anterior wall of the blood vessel and the region apart from the lumen position as a posterior wall of the blood vessel.

### PRIOR ART DOCUMENTS

Patent Document 1: JP-A-2007-283035

However, Patent Document 1 merely proposes the process for setting an ROI on the anterior wall and the posterior wall of a blood vessel and dose not consider the process for setting an ROI corresponding to a plurality of slate points of a contour related to IMT measurement in a blood vessel, therefore accuracy in ROI setting related to IMT measurement still remains as an unsolved problem.

The objective of the present invention is to provide an ultrasonic diagnostic apparatus and the ROI setting method capable of improving accuracy of ROI setting related to IMT measurement.

### BRIEF SUMMARY OF THE INVENTION

In order to achieve the above-described objective, the present invention executes imaging an ultrasonic image of a region including a carotid artery part in an object and sets an ROI region including an intima-media complex based on the degree of concentration of contour slate points in the carotid artery by scanning the ultrasonic image, so as to measure the intima-media thickness in the ROI region.

In concrete terms, the ultrasonic diagnostic apparatus of the present invention scans an ultrasonic image by transmitting/receiving ultrasonic waves to/from a region including a carotid artery of an object and comprises a thickness measurement unit configured to measure thickness of an intima-media complex from the ultrasonic image, further comprising an ROI setting unit configured to scan the ultrasonic image and set an ROI region including the intima-media complex on the ultrasonic image based on the degree of concentration of contour slate points of the carotid artery, wherein the thickness measurement unit measures thickness of the intima-media complex based on the boundary in the set ROI.

Also, the ROI setting method of the ultrasonic diagnostic apparatus related to the present invention executes imaging of an ultrasonic image by transmitting/receiving ultrasonic waves to/from a region including a carotid artery of an object, and includes a first step that measures thickness of an intima-media complex from the ultrasonic image by the thickness measurement unit, comprising a second step that scans the ultrasonic image and sets an ROI region including the intima-media complex on the ultrasonic image based on the degree of concentration of contour slate points of the carotid artery by a region-of-interest setting unit, wherein the first step measures thickness of the intima-media complex based on a boundary in the set ROI region by the thickness measurement unit.

In the above-described configuration of the present invention, accuracy of ROI setting can be improved since an ROI can be set referring to a plurality of concentrated contour slate points (pixel points) by imaging an ultrasonic image by transmitting/ receiving ultrasonic waves to/from a region including a carotid artery in an object, scanning the ultrasonic image and setting an ROI including the intima-media complex based on the degree of concentration of contour slate points of the carotid artery by a region-of-interest setting unit.

### EFFECT OF THE INVENTION

The present invention is effective in providing the ultrasonic diagnostic apparatus and the region setting method capable of improving accuracy of ROI setting in IMT measurement.

### BRIEF DESCRIPTION OF THE DIAGRAMS

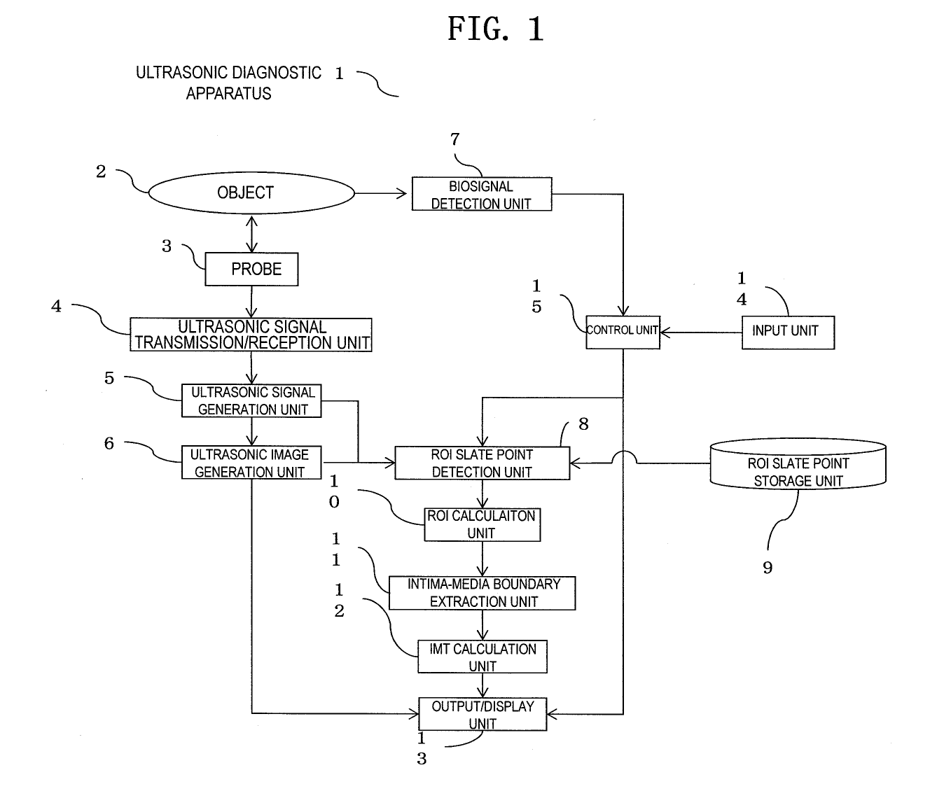

FIG. 1 is a block diagram showing a configuration example of the ultrasonic diagnostic apparatus in a first embodiment of the present invention.

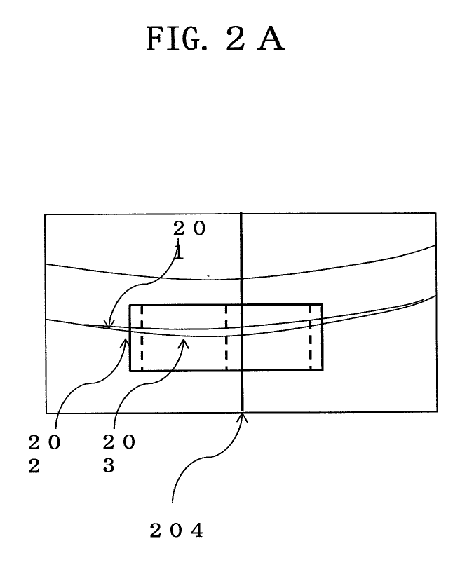

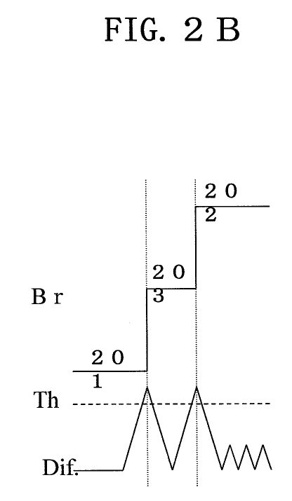

FIG. 2A is a view for explaining the characteristic of brightness change in a carotid wall.

FIG. 2B is a view for explaining brightness change on a line 204 in FIG. 2A.

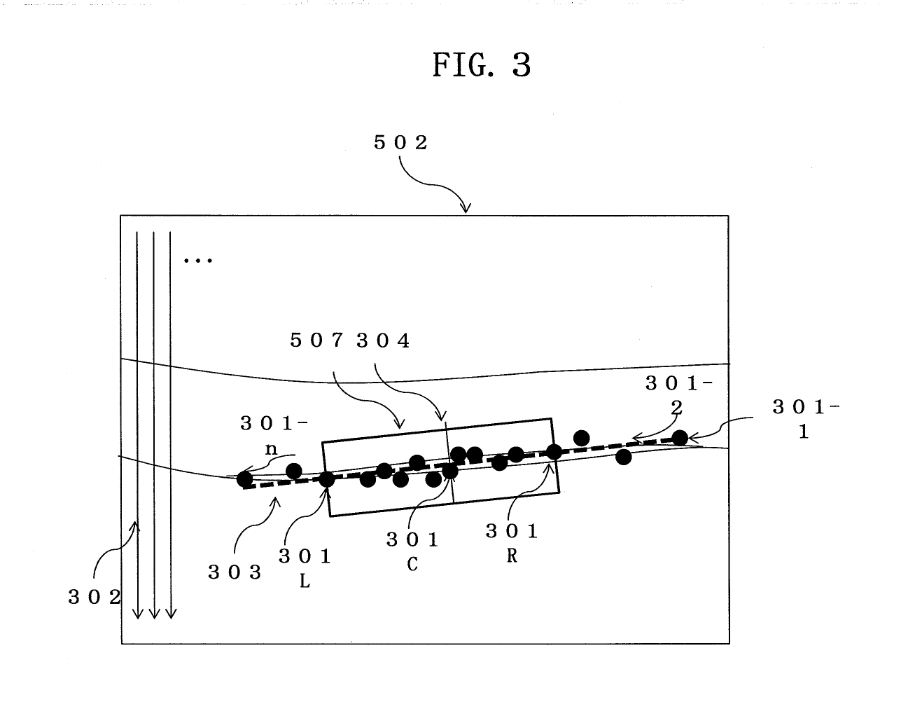

FIG. 3 is a view for explaining the principle of ROI setting in the first embodiment of the present invention.

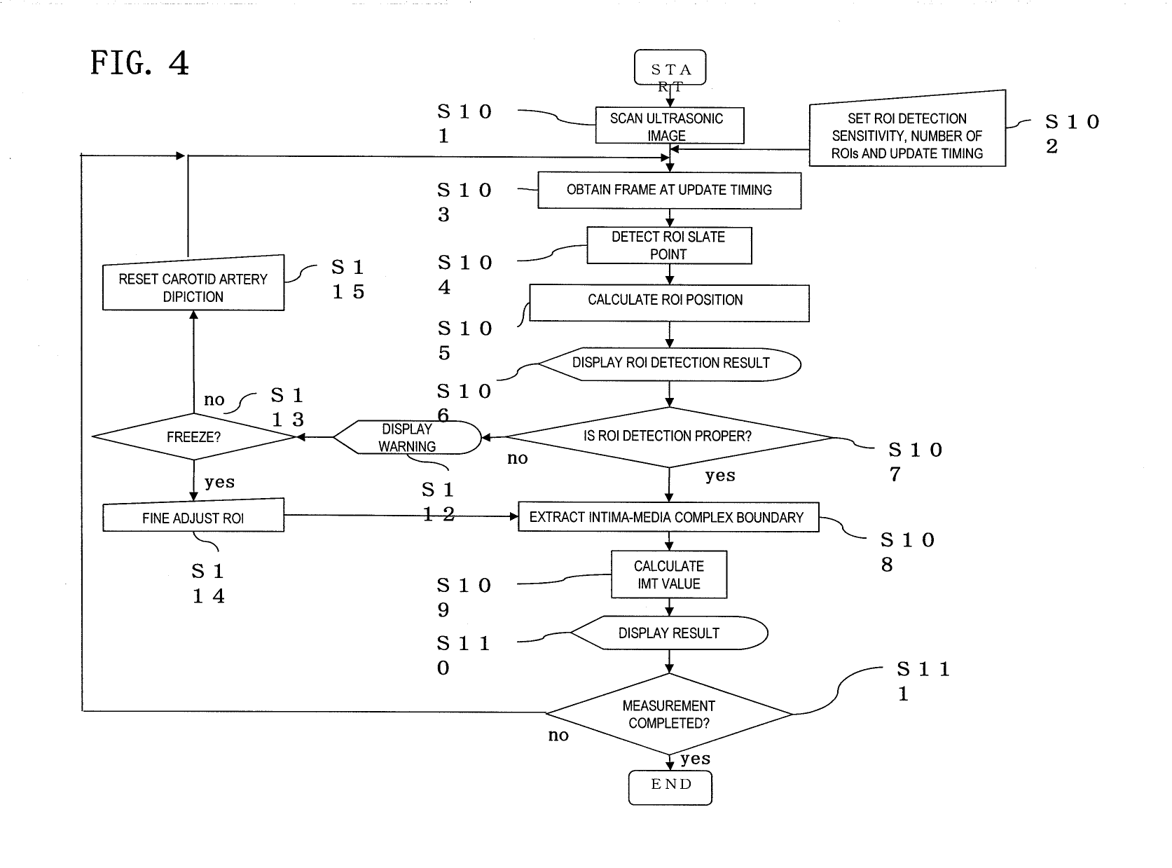

FIG. 4 is a flowchart for explaining an operation example of the ultrasonic diagnostic apparatus in the first embodiment of the present invention.

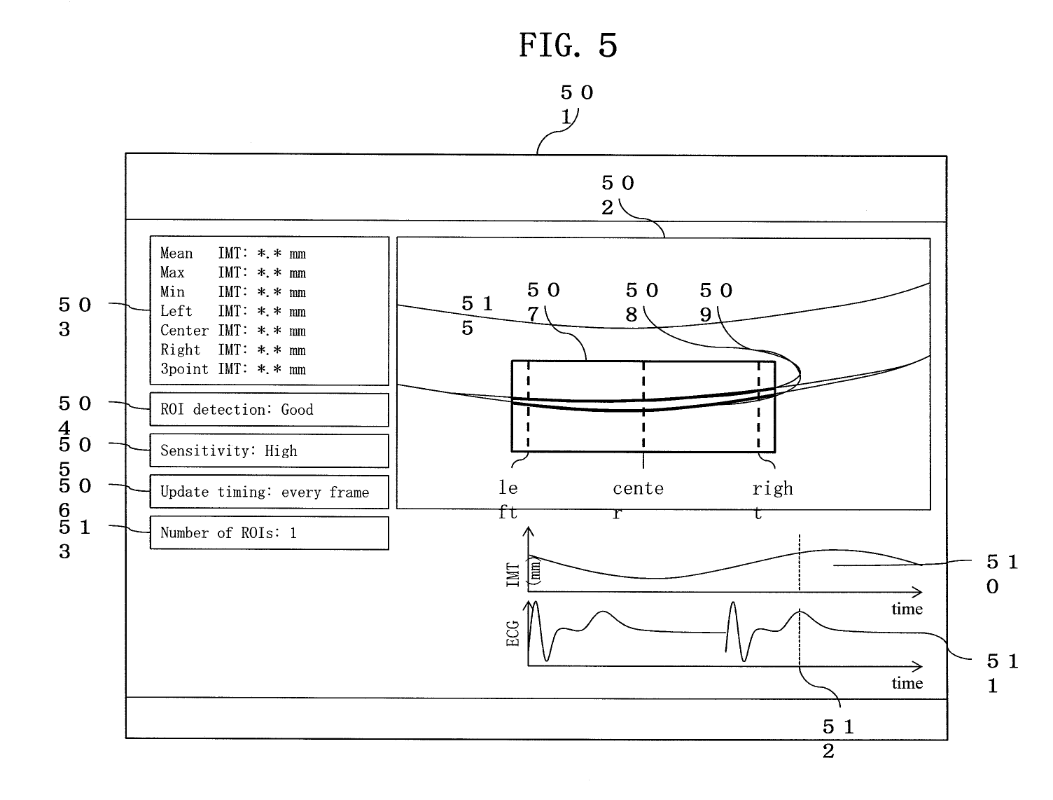

FIG. 5 is a view for explaining a screen display example of the ultrasonic diagnostic apparatus in the first embodiment of the present invention.

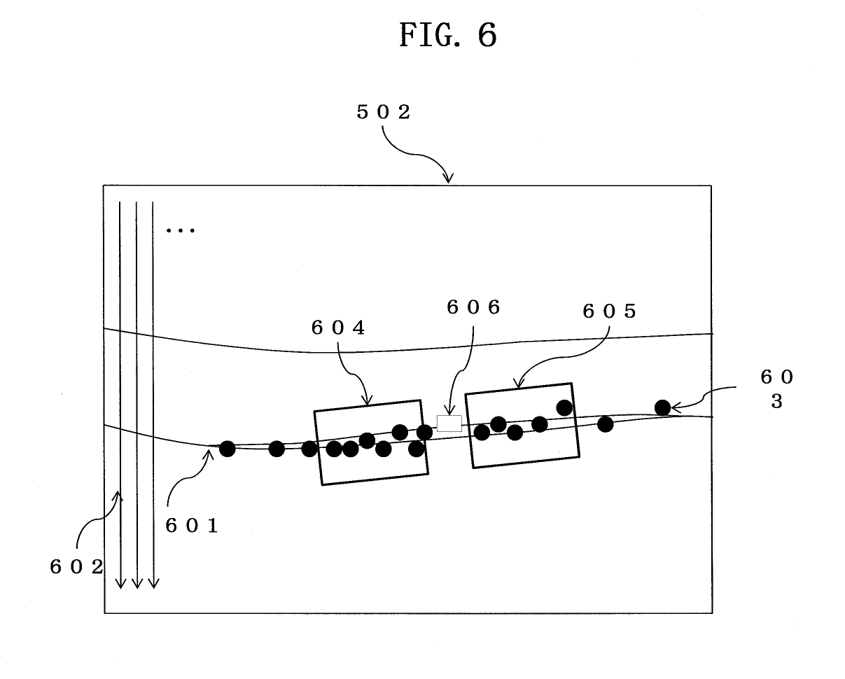

FIG. 6 is a view for explaining the principle of ROI setting in a second embodiment of the present invention.

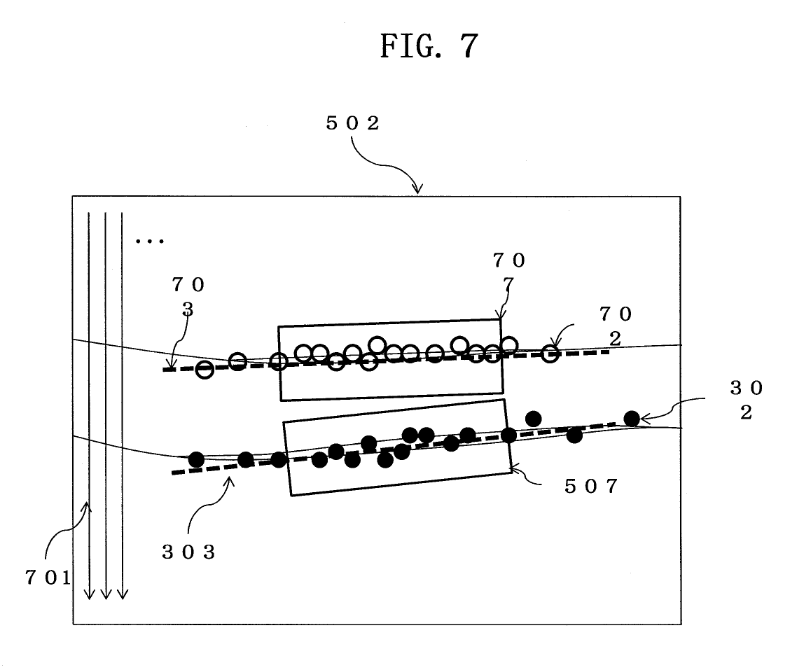

FIG. 7 is a view for explaining the principle of ROI setting in a third embodiment of the present invention.

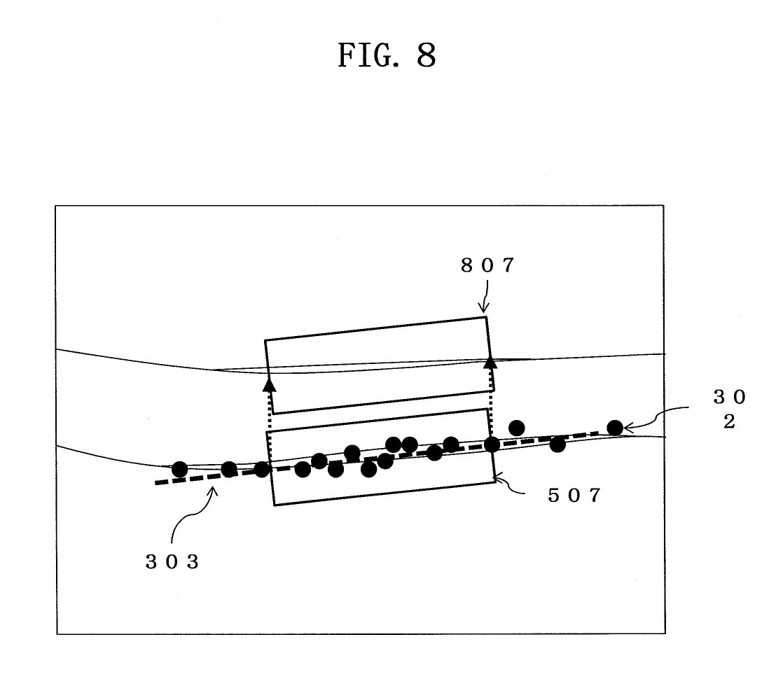

FIG. 8 is a view for explaining an example different from FIG. 7 related to the third embodiment of the present invention.

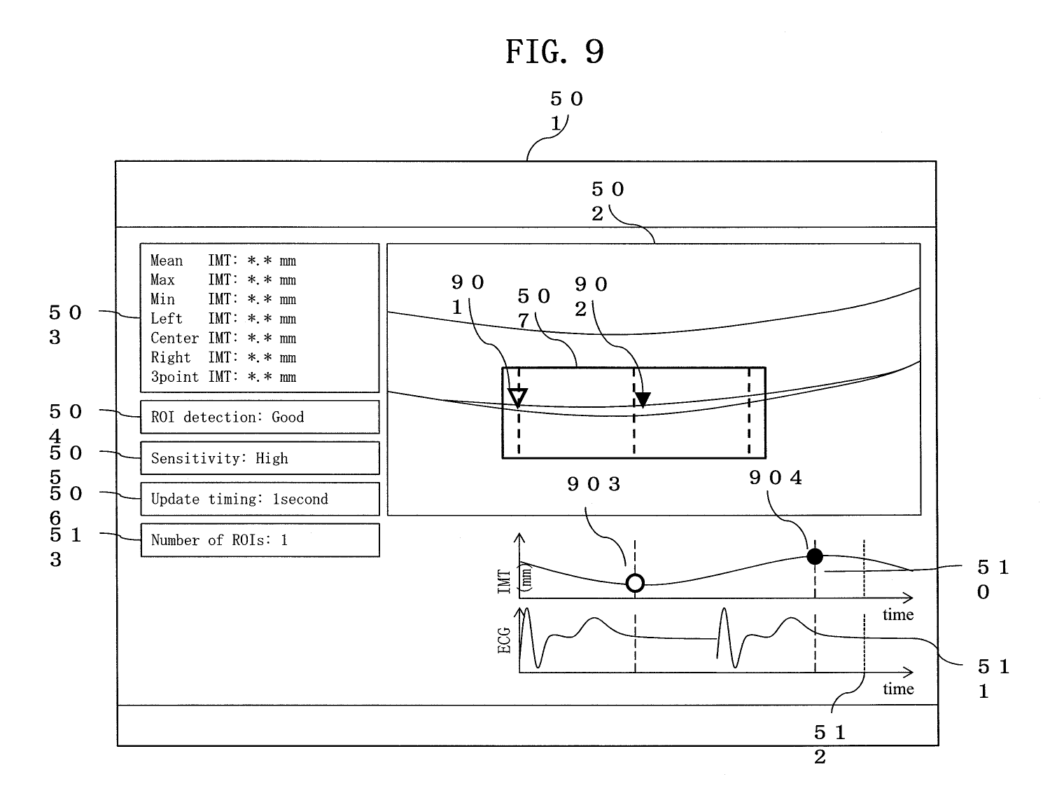

FIG. 9 is a view for explaining a screen display example of the ultrasonic diagnostic apparatus in a fourth embodiment of the present invention.

### DETAILED DESCRIPTION OF THE INVENTION

The first embodiment of the ultrasonic diagnostic apparatus and the ROI setting method to which the present invention is applied will be described below. In the following description, the same function parts are represented by the same reference numerals, and the duplicative description thereof is omitted. The first embodiment exemplifies the case that the number of ROI is one.

### Embodiment 1

FIG. 1 is a block diagram showing the outline of the ultrasonic diagnostic apparatus in the first embodiment of the present invention.

In the first embodiment, an ultrasonic probe 3, an ultrasonic transmission/reception unit 4, an ultrasonic signal generation unit 5 and an ultrasonic image generation unit 6 take on the function to “execute imaging of an ultrasonic image by transmitting/receiving ultrasonic waves to/from a region including a carotid artery of an object”.

Also, an ROI slate point detecting unit 8, an ROI slate point storing unit 9 and ROI calculating unit 10 take on the function to “scan the ultrasonic image and set an ROI region including the intima-media complex on the ultrasonic image based on the degree of concentration of contour slate points of the carotid artery by a region-of-interest setting unit”.

Also, an intima-media complex contour extracting unit 11 and an IMT calculating unit 12 take on the function to “measure intima-media thickness (IMT) from the ultrasonic image by a thickness measurement unit”.

Also, the ultrasonic diagnostic apparatus in the present embodiment has an input unit 14 for an examiner to set ultrasonic imaging condition, etc., a control unit 15 for executing programs to image an ultrasonic image or to measure IMT via setting inputted by the input unit 14 and an output/display unit for outputting/displaying measurement results of the ultrasonic image or the IMT, so as to coordinate the respective functions.

Next, configuration of an ultrasonic diagnostic apparatus 1 in the first embodiment will be described in detail.

The ultrasonic diagnostic apparatus 1 comprises the ultrasonic probe 3, the ultrasonic transmission/reception unit 4, the ultrasonic signal generation unit 5, the ultrasonic image generation unit 6, the biosignal detection unit 7, the ROI slate point detecting unit 8, the ROI slate point storing unit 9, the ROI calculation unit 10, the intima-media boundary extracting unit 11, the IMT calculation unit 12, the output/display unit 13, the input unit 14 and the control unit 15.

The ultrasonic probe 3 transmits/receives ultrasonic waves to/from target tissue in an object via transducers. The kind of ultrasonic probe 3 is categorized mainly by its scanning method such as the linear type, the convex type and the sector type. There is also a case that the ultrasonic probe 3 is merely a probe.

The ultrasonic transmission/reception unit 4 transmits ultrasonic waves to the ultrasonic probe 3 and receives the reflected echo signals from the object via the ultrasonic probe 3.

The ultrasonic signal generation unit 5 executes signal processing on the reflected echo signals from the transmission/reception unit 4 via a phasing circuit or an amplification circuit in accordance with imaging setting of the device so as to acquire the formed ultrasonic signals.

The ultrasonic generation unit 6 generates an ultrasonic image based on imaging setting of the device using the signals inputted from the ultrasonic signals generation unit 5.

The biosignal detection unit 7 detects biosignals of an object 2 and converts them into signal data. Biosignals include ECG (Electro Cardio Gram) or PCG (Phono Cardio Gram), and ECG will be exemplified in the present embodiment.

The ROI slate point detecting unit 8 detects slate points of an ROI on the ultrasonic image generated by the ultrasonic image generation unit 6, using the ROI slate points storing unit 9.

The ROI slate point storing unit 9 stores the slate points for extracting the characteristic of the signal at the position which is adequate for IMT measurement, and operates the ROI slate point detecting unit 8 on the basis of these slate points .

The ROI calculation unit 10 calculates the position and the size of an ROI from the ROI slate point group detected by the ROI slate point detecting unit 8.

The ultrasonic probe 3, the ultrasonic transmission/reception unit 4, the ultrasonic signals generation unit 5 and the ultrasonic image generation unit 6 transmit/receive ultrasonic waves to/from a region including a carotid artery of an object and scan an ultrasonic image thereof.

The intima-media boundary extracting unit 11 and the IMT calculation unit 12 measure thickness of an intima-media complex from the ultrasonic image.

The ROI slate point detecting unit 8, the ROI slate point storing unit 9 and ROI calculation unit 10 scan the ultrasonic image and calculate the position of a region of interest including the intima-media complex based on the degree of concentration of contour slate points in the carotid artery.

Further, the intima-media boundary extracting unit 11 and the IMT calculation unit 12 execute a boundary extracting process of the carotid artery with respect to the region of interest of which the position is calculated, and measure thickness of the intima-media complex from the boundary.

FIG. 2A, FIG. 2B and FIG. 3 will be used for explaining the principle in the process from the detection of ROI slate points to calculation of the position and the size thereof.

First, the process of detecting ROI slate points will be described using FIG. 2A.

FIG. 2A is a view for explaining the characteristic of brightness change in a carotid wall . Though FIG. 2A is actually a B-mode image, only contour lines will be used for explanation.

In the example of a B-mode image of an ultrasonic image, a first characteristic of brightness change is that a lumen 201 which is a bloodstream part of a carotid artery is depicted with low brightness, and an intima-media complex 203 of an intima and a media disposed on the outside of the carotid artery is depicted with higher brightness than the bloodstream.

A second characteristic of brightness change is that an outer membrane 202 disposed on the outside of the intima-media complex 203 of the carotid artery is depicted with even higher brightness than the intima-media complex 203.

Next, an example of analyzing the first characteristic of brightness change and the second characteristic of brightness change will be described using the profile of a line 204 referring to FIG. 2B.

FIG. 2B is a view for explaining brightness change on the line 204 in FIG. 2A.

In FIG. 2B, from the upper left in the diagram, the lumen 201, the intima-media complex 203 and the outer membrane 202 of a carotid artery are disposed in that order. Brightness change Br of the respective regions is indicated by a two-stage process as shown in the diagram.

In other words, the brightness change on the left side in the diagram is shown as the first brightness change, the brightness change on the right side in the diagram is shown as the second brightness change, and the characteristic of the respective brightness changes is stored in the ROI slate point storing unit 9.

A threshold value Th is provided to every change of the first and the second brightness changes. While the threshold value Th in the first brightness change and the second brightness change is set as the same value in FIG. 2B, the threshold value in the first brightness value and the second brightness value can be set arbitrarily.

FIG. 2B shows an example of the case that the value of a differentiation Dif of the first brightness change and the threshold value Th are compared, and a differentiation Dif of the second brightness change and the threshold Th are further compared. The pixel value wherein both values of the differentiation Dif of the first brightness change and the differentiation Dif of the second brightness change are greater than the threshold value is set as ROI slate point data (slate point data 301 in FIG. 3 to be described later).

In the case that the detection can be executed by using one of the first brightness change or the second brightness change, only one of them may be used.

Since the conventional ROI setting is executed by only brightness change without detecting ROI slate points, the brightness change which existing on an image and is similar to an intima-media complex could have been falsely recognized as an ROI . However, if the ROI setting range is specified by slate point data as shown in FIG. 2B, possible false recognition in the conventional ROI setting can be eliminated.

Detection of ROI slate points based on the characteristic of brightness change in an intima-media complex is executed by the above-described procedure.

Next, the process for calculating the position and the size of an ROI will be described referring to FIG. 3. FIG. 3 is a view for explaining the principle of ROI setting in the first embodiment of the present invention.

First, a slate point data 301-1 is plotted on the ultrasonic image in which a coronary artery to be displayed on an image display region 502 of the screen is depicted. Then plural sets of slate point data 301-2˜301-n are acquired by the same procedure, and plural sets of slate point data 301-2˜301-n are plotted on the ultrasonic image in the same manner. The coordinate points of the plotted slate data 301-1˜301-n on the ultrasonic image are stored to be read out in the subsequent process.

Next, the pixel points of the ultrasonic image are scanned, for example in the direction of 302, and a region in the ultrasonic image on which the plotted plural sets of slate point data 301-1˜01-n are unevenly distributed is searched. From the result of searching, the concentration rate of slate point data 301-1˜301-n in one region is searched.

For example, the process for acquiring the degree of concentration is, to first define a regression line 303 which passes through the maximum number of slate point data 301-1˜301-n. The start point and the endpoint of the regression line 303 passes through the edge points in the left-and-right direction of the distributed slate point data 301-1˜301-n. In other words, the position where the regression line 303 exists is the point where the slate point data 301-1˜301-n are concentrated, which is the index of concentration ratio.

Here, the index for the degree of concentration is defined, when plural sets of 2-dimensional coordinate points (xi, yj) of the slate point data exist, by the length of a distance between the respective coordinate points and a threshold.

Also, the threshold value is obtained by, for example an average value μ which is zero and a standard deviation σ. In other words, the index of concentration ratio is respectively defined that −σ+μ˜+σ+μ (68%) is the range of value which is most concentrated (best value range), −2 σ+μ˜+2 σ+μ (95%) is the range of value which is more concentrated (better value range) and −3 σ+μ˜+3 σ+μ (99%) is the concentrated range of value (good value range) . The best value range, the better value range and the good value range may be defined also as, for example a narrow value range, a wider value range and a further wide value range as far as they are defined by three stages, without being limited to an average value μ and a standard deviation σ.

Then the shape of the ROI 507 is set as a rectangle, and the position of the ROI 507 is set, for example at the center of a point 301C where the slate point data 301-1˜301-n is most concentrated on the regression line 303. The point 301C can be selected by an examiner from among the best value range, the better value range and the good value range using the input unit 14 according to the condition of image quality, etc. of the carotid artery image obtained from the object.

Also, a point 301L which is at the farthest left among the slate point data 301-1˜301-n and a point 301R at the farthest right thereof plotted on the regression line 303 are set as the ends of the ROI 507.

Calculation of the slate point data 301C is executed by comparing the respective coordinate values of the stored coordinates of the slate point data 301-1˜301-n on the image, and setting the slate point data 301-1˜301-n having the most number of approximated coordinate values, i.e. which is at the nearest coordinate point to the concentrated coordinate values, as the slate point data 301C based on the comparison result. The position and the size of the ROI 507 in the direction parallel to the regression line 303 are determined by the above-described method.

Next, the position and the size in the direction vertical to the regression line 303 is determined by, for example drawing a vertical line 304 of the regression line 303 in the direction passing through the slate point data 301C, acquiring the maximum range between the slate point data 301c passing through the vertical line 304 and the other slate point data, and calculating the function related to the acquired maximum range (double here). The direction vertical to the regression line 303 is also calculated by the above-described method, thus the position and the size of the ROI 507 is determined.

While the case that a rectangle shape is used for an ROI profile of an intima-media complex is exemplified in the present embodiment, an arbitrary shape besides a rectangle may also be used.

Also, the size of an ROI may be set as the width of standard deviation of the coordinate value of the slate point data 301-1˜301-n in the direction of the regression line 303 and the direction vertical thereto.

In another method, by assuming that density distribution of the ROI slate point group is subjected to a certain 2-dimensional distribution and presuming the density distribution, the center of the 2-dimensional distribution may be set as the center of an ROI, the extensity may be set as the size, and the direction of extensity may be set as the gradient of the ROI.

The ROI slate point storing unit 9 may be set so that the sensitivity related to the signal noise ratio can be adjusted. For example, the extent of allowing the influence of noise, etc. is adjusted by setting the threshold value to the brightness derivative value or the lumen-side brightness value. A concrete example of adjustment is that when the threshold value with respect to the brightness derivative value is set as a small value the influence of noise is tolerated and an intima-media complex can be detected even when it is somewhat indistinct.

Also, when the threshold value with respect to the lumen-side brightness derivative value is set as a small value, an intima-media complex can be detected even when noise on the lumen side is great. This setting is operated by an examiner using the input unit 14.

The intima-media complex extracting unit 11 extracts the lumen side boundary and the outer-membrane side boundary within an ROI . The boundary extracting unit 12 extracts the lumen side boundary and the outer-membrane side boundary based on the position of a parting line. For example, the boundary extracting unit 12 extracts the lumen side boundary within the range limited toward the lumen side from the lumen side parting line, and also extracts the outer-membrane side boundary within the range limited toward the outer-membrane side from the outer-membrane side parting line. The kind of the boundary extracting method to be used here is based on a limited search, the edge detection, the region growing, an active contour model, and so on.

The boundary extracting unit 12 executes boundary extracting method based on the limited search by limiting the range of the lumen side boundary of an intima-media region to the lumen side. Also, it extracts the outer-membrane side boundary by limiting the range to the outer-membrane side. The processing by this boundary extracting method is applied to the entire rows in an ROI for extracting the boundary. The groups of these positions are set as the lumen side boundary and the outer-membrane side boundary.

The extracting method based on the edge detection calculates the contour of an object by the change of brightness in an image. Also, the contour here means the place where the brightness in an image changes drastically, i.e. the boundary of the region in the object.

The region growing method uses a threshold value by applying the fact that “the region within the same tissue has a small brightness change” and that “brightness change occurs between different tissues”, and executes enlargement (or reduction) of a region while taking in the interconnecting pixels from the target region so as to extract the entire target region.

The boundary extracting method based on an active contour model is the image region extraction by an active contour represented by a Snake. The characteristic of the boundary extracting method based on an active contour model is that it is resistant to noise and is capable of acquiring smooth and continuous contours.

The IMT calculation unit 12 calculates the distance between the boundary on the side of the lumen 201 and the boundary on the side of the outer-membrane 202. The calculated value is the IMT. Further, the IMT calculation unit 12 calculates values such as the average value, the maximum value and the minimum value in an ROI.

The output/display unit 13 outputs the boundary or measured values to a measurement report or displays them on the display region 502 on a screen of the ultrasonic diagnostic apparatus 1. The concrete examples of the output/display unit 13 are referred to as an image display unit such as a liquid crystal monitor and a CRT monitor.

The input unit 14 is user interface for an examinee to execute initial setting for IMT measurement or manual operation for correcting the position of a detected ROI. The concrete examples of the input unit 14 are devices such as a keyboard, a trackball, a mouse or a switch.

The control unit 15 controls the entire system. In concrete terms, the control unit 15 receives the information on power or timing of signal transmission/reception and controls the ultrasonic transmission/reception unit 4 to acquire desired ultrasonic signals. Also, the control unit 15 generates a timing signal which operates the ROI slate point detecting unit 8 from the phase of a biosignal outputted from the biosignal detecting unit 7. The concrete example of the control unit 15 is a device such as a CPU.

Next, an operation example of the ultrasonic diagnostic apparatus in the first embodiment related to the present invention will be described using FIG. 3, FIG. 4 and FIG. 5.

FIG. 4 is a flowchart showing an operation example of the ultrasonic diagnostic apparatus in the first embodiment related to the present invention, and FIG. 5 is a view for explaining a screen display example of the ultrasonic diagnostic apparatus in the first embodiment related to the present invention.

Display regions or display items of numeral references 501˜515 in FIG. 5 will be described below since the flowchart shown in FIG. 4 will be described referring to a display example in FIG. 5.

501 shows a display screen. 502 is an image display region in the display screen 501, in which an ultrasonic image of a carotid artery is displayed. 503 is a display region of IMT values, in which the average, maximum, the minimum, the left edge, the center, the right edge and the average of three points (the left edge, the center and the right edge) are displayed by numeral values. 504 is a display region of detection state of an ROI, and a term such as “good” or “bad” is displayed therein.

505 is a display region of detection sensitivity, wherein the sensitivity is set as “high” when a lumen portion of a carotid artery or an outer region of a blood vessel wall is depicted clearly or as “middle” when a lumen portion of a carotid artery or an outer region of a blood vessel wall is depicted not very clearly due to the case of advanced arterial sclerosis. 506 is a display region of detection timing, and “every frame” is selected in FIG. 5. 507 shows an ROI, 508 shows a lumen side boundary and 509 shows an outer-membrane side boundary respectively.

510 shows a time passage curve of an IMT value, 511 shows a time passage curve of ECG and 512 shows a time phase marker for selecting a certain time phase of ECG respectively. 513 is a display region of the number of ROIs, and “1” is exemplified as the number of ROIs in FIG. 5.

Next, an operation example of the ultrasonic diagnostic apparatus in the first embodiment related to the present invention will be described using FIG. 4.

An examiner applies the ultrasonic probe 3 on a cervical region of an object 2, and images an ultrasonic image of a carotid artery. The ultrasonic image is displayed on the image display region 502 (S101).

The examiner sets detection sensitivity, the number and the update timing of an ROI using the input unit 14. The detection sensitivity, the number and the update timing of an

ROI are displayed on display regions 505, 506 and 513, and “High” is displayed on the display region 505, “1” is displayed on the display region 506 and “every frame” is displayed on the display region 513 (S102).

The control unit 15 causes the ultrasonic image generation unit 6 to obtain a frame image of the update timing (“every frame” here) set in S102 (S103).

The control unit 15 causes the ROI slate point detecting unit 8 to detect the ROI slate points on the ultrasonic image obtained in S013 (S104) . In concrete terms, the ROI slate point detecting unit 8 scans the pixel points in the ultrasonic image in the direction of 302 as shown in FIG. 3, and searches a partial region of the ultrasonic image where plural sets of slate point data 301-1˜301-n are unevenly distributed which are plotted. As a result of search, the degree that slate point data 301-1 301-n are concentrated in one region on the ultrasonic image (concentration ratio) is inspected.

The control unit 15 controls so that the position and the size of the ROI 507 are set using the ROI slate points detected in 5104 (S105) . Concretely, the position and the size of the ROI 507 is set in the direction parallel and vertical to the regression line 303 described in FIG. 3.

The control unit 15 controls so that the ROI 507 set in S105 is superimposed over the ultrasonic image of a carotid artery displayed on the image display region 502 in the display screen 501 of the output/display unit 13 (S106).

The control unit 15 determines whether or not setting of the ROI 507 in S105 is executed properly. For example, assuming that the reference number of ROI slate points is 100, when the ROI slate points wherein the number thereof is deviated from the range of plus and minus 10% of the reference number are measured, for example 89 or less or 111 or more, such condition is determined as non-detective state of an ROI.

Also, other than the number of ROI slate points stored in the ROI slate point storing unit 9, the calculated reference values of the central position, extensity and gradient of the ROI can be used for the determination. Whether or not an ROI is properly set is displayed by coloring a frame border of the ROI 507 for distinction. For example, the color of the frame border of the ROI 507 is set as green when an ROI detection result is proper, and the color thereof is set as red when it is improper.

Also by quantifying the ROI detection result using the numeric values of the central position, extensity and angle of the ROI 507, the color of the frame border of ROI 507 may be consecutively changed from green color to red color according to the numeric values.

Also, besides changing the color in the frame border of the ROI 507, the frame border may be depicted as a solid line when ROI detection is properly executed, and the frame border may be depicted as a dotted line or a blinking line when ROI detection is improperly executed. In this manner, it is easy to visibly confirm condition of the ROI detection. Also, detection state (information on whether proper or improper) can be displayed in the ROI detection state display 504 on the display screen 501 (S107).

The control unit 15 causes the intima-media boundary extracting unit 11 to extract a boundary of an intima-media complex with respect to the pixels included in the ROI 507 which is determined as proper in S107 (S108).

The control unit 15 causes the IMT calculation unit 12 to calculate the IMT value from the boundary of the intima-media complex extracted in S108 (S109).

The control unit 15 makes the IMT value calculated in S109 to be displayed by numeric value on the IMT value display region 503 in the display screen 501 of the output/display unit 13.

Also, the control unit 15 makes the IMT value calculated in S109 to be displayed by the time passage curve 510 of the IMT value on the display screen 501 of the output/display unit 13 (S110).

The examiner inputs whether IMT measurement is completed or not using the input unit 14. The control unit 15 receives the determination of completion of IMT measurement inputted by the input unit 14, completes the program if the result indicates completion, ends the program if the IMT measurement is completed and executes the process of 5103 if the result indicates that IMT measurement is not completed (S111).

Next, in the case that detection is not proper in S107, the control unit 15 changes the display items and display pattern by changing the color or line pattern of the frame border in the ROI, and displays the message “bad” on the display region 504 of ROI detection state as a warning (S112).

There are cases that the control unit 15 determines the display region 504 of ROI detection state as “Bad” but it can be shifted to be used for IMT calculation by fine-adjusting the ROI position, as a result of image observation by the examiner. There are also cases that the detection sensitivity, quantity and the update timing of ROIs need to be reset by the examiner using the input unit 14.

Given this factor, as an optional function for a case that shifting of an ROI to fine-adjustment is possible, the control unit 15 determines whether or not to freeze acquisition of the ultrasonic image. As a result of determination, S114 is carried out when freezing of image acquisition is to be executed so as to fine-adjust the position or the size of an ROI, and S115 is carried out when input for resetting to a carotid artery image is necessary (S113).

By the freezing process in S113, the examiner fine-adjusts the position or the size of an ROI using the input unit 14 and shifts to S108 (S114).

On the other hand, the examiner does not execute freezing in S113, resets the detection sensitivity, the number and the update timing of an ROI using the input unit 14 (S115), and carries out the process of S103.

In accordance with the above-described present embodiment, an ROI is set referring to contour slate point, i.e. plural pixel points, whereby it is possible to improve accuracy of ROI setting. Also, the specific effect of the present embodiment is that an examiner can make fine adjustment of ROI by input setting even when the ROI setting is determined as no good, which leads to improvement of operationality.

### Embodiment 2

The second embodiment exemplifies the case that there are two or more ROIs. Since the configuration and operation of the ultrasonic diagnostic apparatus 1 is the same as the first embodiment, the description thereof will be omitted and only different parts will be described.

The calculation step of the position and the size of an ROI will be described referring to FIG. 6.

FIG. 6 is a view explaining the principle of ROI setting in the second embodiment of the present invention.

First, slate data 603 is plotted on the ultrasonic image in which the carotid artery to be displayed on the image display unit 502 in a screen is depicted. Then plural sets of the slate point data 603 are acquired by the same procedure, and plotted in the same manner on the ultrasonic image. The coordinate points of the plotted slate point data 603 on the ultrasonic image are stored to be read out in the subsequent process.

Next, the control unit 15 scans the pixel points of the ultrasonic image in the direction of, for example 602 and searches the region where plural sets of the plotted slate point data 603 are unevenly distributed on the ultrasonic image.

In the present embodiment, the case that there are two searched regions that are not consolidated. In addition, the process can be performed in the same manner even when there are three or more searched regions.

First, the control unit 15 extracts a contour 601 of the blood vessel wall in a carotid artery and stores the position of the contour 601 in the blood vessel wall on the ultrasonic image. Then the control unit 15 calculates and outputs the segment wherein more than a predetermined number (five here) of the slate point data 603 are detected on the stored contour 601 of a blood vessel as the ROI 604 and the ROI 605. The position and the size of the ROI 604 and the ROI 605 in the direction along the contour 601 are determined by the above-described process. Another method, in the case that the contour 601 is not extracted, sets an ROI in the same manner as the first embodiment by calculating the regression line of the slate point data 603 included in the segment in which more slate point data 603 than a predetermined number is detected.

Next, the control unit 15 acquires the position and the size of the contour 601, the ROI 604 and the ROI 605 in the normal line direction by, for example drawing a vertical line at the respective midpoints of the ROI 604 and the ROI 605 in the direction along the contour 601, acquiring the maximum range between the slate point data passing through the respective vertical lines, and further acquiring integral multiplication (double here) of the acquired maximum range. The contour 601 and the normal line direction are also determined by the above-described process, thus the position and the size of the ROI 604 and the ROI 605 are determined.

In IMT value calculation, an IMT value maybe calculated using plural ROIs or the average value of the IMT values of plural ROIs may be calculated. The IMT value and the ultrasonic image are outputted and displayed on the output/display unit 13 as in the first embodiment.

Also, it is easier for the examiner to distinguish between ROIs and the regions that are not an ROI by changing the display pattern of a region 606 between the ROI 604 and the ROI 605 which is not recognized as an ROI by changing the color or gradation thereof from that of the other regions.

Also, there are cases that the ROI 604, the ROI 605 and the region 606 are observed by the examiner and they can be combined as one ROI 507 as shown in the first embodiment. When the case can be determined as appropriate combine the regions, the examiner inputs the command to the input unit 14 to consolidate the ROI 604, the ROI 605 and the region 606 into one region as ROI 507. The control unit 15 receives the input of command for making one ROI, consolidates the ROI 604, the ROI 605 and the region 606 to generate one ROI such as the ROI 507 in the first embodiment.

In accordance with the above-described present embodiment, an ROI is set referring to contour slate points, i.e. plural pixel points, thus accuracy of ROI setting can be improved.

Also, the specific effect of the present embodiment is that, even when an intima-media complex is depicted while being discontinued, the measurement values can be calculated by setting plural ROIs and executing boundary extraction only in the positions where boundary extraction can be executed.

Since the region where an ROI is not extracted avoids the region where error can be easily caused upon boundary extraction of an intima-media complex, not only improvement of boundary extraction but also improvement of accuracy in IMT value measurement can be expected.

Also even when plural ROIs are recognized, calculation of the position and the size of the respective ROIs can be more simplified compared to the first embodiment.

Even when there are separate plural ROIs, since it is possible to consolidate them into one ROI by an examiner's observation, the effect in the first embodiment can be exerted after the consolidation.

### Embodiment 3

The third embodiment explains an example that the ROI setting executed on the blood vessel wall of a carotid artery which is closer to an ultrasonic probe (one side) is reflected on the ROI setting of the blood vessel which is farther from the probe (the other side).

Since the configuration and operation of the ultrasonic diagnostic apparatus 1 is the same as the first embodiment, the description thereof will be omitted and only the parts different from the first embodiment will be described.

The calculation step of the position and the size of an ROI in the present embodiment will be described using FIG. 7.

FIG. 7 is a view for explaining the principle of ROI setting in the third embodiment of the present invention.

First, data processing is executed on one side of the carotid artery which is an outer wall part in the lower side of the diagram, as in FIG. 3.

Next, the following data processing is executed on the other side of the carotid artery which is the outer wall part in the upper side of the diagram.

Slate point data 702 is plotted on the ultrasonic image in which a carotid artery to be displayed on the image display region 502 of a screen is depicted. Then plural sets of slate point data 702 are acquired in the same procedure and plotted in the same manner on the ultrasonic image. The coordinate points of the plotted slate point data 702 on the ultrasonic image are stored to be read out in the subsequence process.

Next, the pixel points on the ultrasonic image are scanned, for example in the direction of 701, and a partial region of the ultrasonic image where the plotted plural sets of slate point data 702 are unevenly distributed is searched. From the result of search, the degree that the plural sets of slate point data 702 are concentrated (concentration ratio) is searched.

The concentration ratio is acquired, for example by first defining the regression line 703 which passes through the maximum number of the slate point data 702. The start point and the end point are set as the ends of the distributed slate point data 702 in the horizontal direction. In other words, the position where there is the regression line 703 is the position where the plural sets of slate point data 702 are concentrated, which is the index of the concentration ratio.

Then the profile of the ROI 707 is set as a rectangle, and the position and the size of the ROI 707 is determined on the regression line 703 as described in the first embodiment.

While a rectangle is exemplified as the ROI profile of an intima-media complex in the present embodiment, an arbitrary profile besides a rectangle may be used.

The position and the size of an ROI of the outer wall part on one side and an ROI of the outer wall part on the other side of a carotid artery can be set by the above-described procedure. However, it is difficult to execute quantitative IMT measurement if the position and the size of the set ROIs are different.

Given this factor, if the position and the size of an ROI of the outer wall part on one side of a carotid artery are different from an ROI of the outer wall part on the other side, the position and the size of the ROI on one side of the carotid artery is matched to those of the ROI on the other side.

For example, the area ratio between the area of one ROI and the area of the other ROI is acquired, and if the area ratio is within plus or minus 10%, the IMT value is calculated as usual from one ROI and the other ROI.

In the case that the area ratio deviates from the range of plus or minus 10%, one ROI is recalculated in accordance with the area of the other ROI, and the IMT value is calculated from the recalculated respective ROIs.

In concrete terms, if the area of one ROI is 200 cm², the IMT value can be calculated as usual when the area of the other ROI is 180˜220 cm².

However, when the area of the other ROI is deviated from 180˜220 cm², the area of the other ROI is adjusted to make the area of the other ROI to fall in the range of 180˜220 cm². Here, selection to match the areas of ROIs can be executed by an examiner by inputting the information to input unit 14 and by the control unit 15 which receives the inputted information.

Also, there is a simpler method which can be described referring to FIG. 8.

FIG. 8 is a view for explaining another example which differs from the example shown in FIG. 7.

In FIG. 8, the frame border of the ROI 507 set on the outer wall part of a carotid artery shown in the lower part of the diagram is copied on the frame border of an ROI 807 of the outer wall part in the carotid artery shown in the upper part of the diagram.

The contour of the outer wall part in the carotid artery shown in the upper part of the diagram is extracted in advance, and the frame border of the ROI 807 is shifted on the contour of the outer wall part of the carotid artery shown in the upper part of the diagram. In the case of not extracting a contour, the frame border may be manually shifted using the input unit 14.

In accordance with the above-described copying process, there is no need to adjust the areas of the ROI 507 and the ROI 807 since the areas of the ROI 507 and the ROI 807 are the same.

In accordance with the above-described present embodiment, ROI setting can be improved since an ROI is set referring to contour slate points, i.e. plural pixel points.

Also, the specific effect of the present embodiment is that ROIs of the outer wall part of a carotid artery in the lower part of the diagram and the outer wall part in the upper part in the diagram can be set at the same time, whereby improving operationality in ROI setting for an examiner.

Also, the present embodiment can eliminate the process of adjusting the size of each ROI by copying the ROI of the outer wall part in a carotid artery shown in the lower part of the diagram to the ROI of the outer wall part shown in the upper part of the diagram, it is possible to improve operationality in ROI setting for an examiner.

### Embodiment 4

The fourth embodiment describes an example of displaying the maximum value or the minimum value of an IMT measurement value while being associated with the set ROI.

Since the configuration and operation of the ultrasonic diagnostic apparatus 1 is the same as the first embodiment, the description thereof will be omitted and only different parts from the first embodiment will be described.

The step of displaying the maximum value or the minimum value of an IMT value in the present embodiment will be described using FIG. 9.

FIG. 9 is a view for explaining a screen display example of the ultrasonic diagnostic apparatus in the fourth embodiment related to the present invention. Only the reference numerals besides those described in FIG. 5 will be described in FIG. 9.

901 shows the minimum value of an IMT in the ROI 507, 902 shows the maximum value of IMT in the ROI 507, 903 shows the minimum value of the time passage curve in an IMT and 904 shows the maximum value of the time passage curve in IMT.

While a maximum value 902 of IMT is indicated by ▾, a minimum value 901 of IMT is indicated by ∇, a maximum value 904 of the time passage curve in IMT is indicated by and the minimum value of the time passage curve in IMT is indicated by O, colors or profiles do not have to be limited for indicating the position of the maximum values and the minimum values.

Further, in the case that the maximum value of the measured IMT is greater than the standardized IMT value for healthy subjects, the mark may be, for example a triangle colored in red to make it distinctive.

In this manner, it is easier to visually recognize the position of the maximum value and the minimum value of IMT or abnormality thereof.

In accordance with the above-described present embodiment, accuracy in ROI setting can be improved since an ROI is set referring to contour slate points, i.e. plural pixel points.

Also, the specific effect of the present embodiment is that the maximum value and the minimum value of IMT or abnormality thereof are easy to confirm, which leads to improvement in diagnosis performance by an examiner.

The preferable embodiments of the ultrasonic diagnostic apparatus, etc. according to the present invention have been described referring to the attached diagrams. However, the present invention is not limited to these embodiments. It is obvious that persons skilled in the art can make various kinds of alterations or modifications within the scope of the technical idea disclosed in this application, and it is understandable that they belong to the technical scope of the present invention.

### DESCRIPTION OF REFERENCE NUMERALS

1: ultrasonic diagnostic apparatus
2: object
3: ultrasonic probe
4: ultrasonic transmission/reception unit
5: ultrasonic signal generation unit
6: ultrasonic image generation unit
7: biosignal detection unit
8: ROI slate point detecting unit
9: ROI slate point storing unit
10: ROI calculation unit
11: intima-media boundary extracting unit
12: IMT calculation unit
13: output/display unit
14: input unit
15: control unit

1. An ultrasonic diagnostic apparatus that images an ultrasonic image by transmitting/receiving ultrasonic waves to/from a region in an object to be examined including a carotid artery portion, having a thickness measuring unit configured to measure thickness of an intima-media complex from the ultrasonic image, which comprises a region-of-interest setting unit configured to scan the ultrasonic image and set a region of interest including the intima-media complex based on the concentration degree of contour slate points in the carotid artery on the ultrasonic image, wherein the thickness measuring unit measures thickness of the intima-media complex based on the boundary in the set region of interest.

2. The ultrasonic diagnostic apparatus according to claim 1, wherein the region-of-interest setting unit calculates position of the region of interest by performing derivation on a first brightness change in an outer-membrane and an intima-media of the intima-media complex and on a second brightness change in an intima-media and a lumen respectively.

3. The ultrasonic diagnostic apparatus according to claim 1, wherein the region-of-interest setting unit calculates position of the region of interest based on the brightness of a bloodstream part in the carotid artery.

4. The ultrasonic diagnostic apparatus according to claim 1, wherein the region-of-interest setting unit, in the case that there are plural aggregations of pixels, calculates position of the region of interest for each of the aggregation using the degree of concentration.

5. The ultrasonic diagnostic apparatus according to claim 4, wherein the region-of-interest setting unit consolidates the region of interests that are calculated for the respective aggregations, and calculates position of one region of interest.

6. The ultrasonic diagnostic apparatus according to claim 1, comprising a display unit configured to display regions in which the position of a region of interest can not be calculated by the region-of-interest setting unit, by changing the display pattern thereof from that of the other regions.

7. The ultrasonic diagnostic apparatus according to claim 1, wherein the region-of-interest setting unit calculates position of a region of interest in a blood vessel wall on one side of the carotid artery based on the position of a region of interest in a blood vessel wall on the other side of the carotid artery.

8. The ultrasonic diagnostic apparatus according to claim 7, wherein the region-of-interest setting unit calculates position of a region of interest in the blood vessel wall on the other side of the carotid artery referring to the concentration degree of contour slate points of the blood vessel wall on the other side of the carotid artery.

9. The ultrasonic diagnostic apparatus according to claim 7, wherein the region-of-interest setting unit calculates position of a region of interest in the blood vessel wall on the other side of the carotid artery by copying the information on a region of interest in the blood vessel wall on one side of the carotid artery.

10. The ultrasonic diagnostic apparatus according to claim 1 comprising:
a thickness calculating unit configured to calculate the maximum value or the minimum value in the thickness of the intima-media complex;
a marker generating unit configured to generate a marker which indicates the thickness of the maximum value or the minimum value; and
a display unit configured to display the mark, the intima-media thickness and the ultrasonic image.

11. A region-of-interest setting method of the ultrasonic diagnostic apparatus which images an ultrasonic image by transmitting/receiving ultrasonic waves to/from a region in an object to be examined including a carotid artery portion, including a first step of measuring thickness of an intima-media complex on the ultrasonic image by a thickness measuring unit, comprising a second step of scanning the ultrasonic image and setting a region of interest including the intima-media complex on the ultrasonic image based on the concentration degree of contour slate points of the carotid artery by the region-of-interest setting unit, wherein the first step measures thickness of the intima-media complex by the thickness measuring unit based on the boundary in the set region of interest.

12. The region-of-interest setting method according to claim 11, including a third step of displaying regions in which position of the region of interest can not be calculated by the display unit, by changing the display pattern thereof from that of other regions.

13. The region-of-interest setting method according to claim 11, including a fourth step of calculating position of a region of interest in a blood vessel wall on one side of the carotid artery based on the position of a region of interest in a blood vessel on the other side of the carotid artery by the region-of-interest setting unit.

14. The region-of-interest setting method according to claim 11, comprising:
a fifth step of calculating the maximum value or the minimum value in the intima-media thickness by a thickness calculating unit;
a sixth step of generating a marker which indicates the maximum value or the minimum value by a marker generating unit; and
a seventh step of displaying the mark, the intima-media thickness and the ultrasonic image by a display unit.
